(19) United States
(12) Patent Application Publication
(10) Pub. No.: US 2002/0156068 A1
(43) Pub. Date: Oct. 24, 2002

(54) Anti-psychosis combination

(76) Inventors: Dominic P. Behan, San Diego, CA (US); Derek T. Chalmers, Cardiff, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/104,602

(22) Filed: Mar. 22, 2002

Related U.S. Application Data
(60) Provisional application No. 60/278,516, filed on Mar. 22, 2001.

(51) Int. Cl. A61K 31/551; A61K 31/519; A61K 31/445; A61K 31/415

(52) U.S. Cl. 514/220; 514/259.41; 514/317; 514/406

(57) ABSTRACT

This invention relates to methods of reducing hyperlocomotor activity and stereotypy by administering a composition comprising a modulator of the 5-HT2A receptor with a neuroleptic agent used for treating psychoses, such as Haloperidol. The invention further relates to compositions comprising a modulator of the 5-HT2A receptor with a neuroleptic agent.

The present application claims priority benefit of Application Serial No. 60/278,516, filed Mar. 22, 2001, the disclosure of which is hereby incorporated by reference in its entirety.

### FIELD OF THE INVENTION

The present invention relates to methods of reducing hyperlocomotor activity and stereotypy by co-administering a modulator of the 5-HT2A serotonin receptor having inverse agonist properties at receptor, preferably N-[3-(4-bromo-2-methylpyrazol-3-yl)phenyl] [(4-chlorophenyl)amino]carboxamide or a derivative thereof, with a neuroleptic agent used for treating psychoses, such as Haloperidol. The present invention also relates to compositions, including pharmaceutical compositions, comprising a modulator of the 5HT-2A receptor and a neuroleptic.

### BACKGROUND OF THE INVENTION

Serotonin, the endogenous ligand for the 5-HT receptor, is thought to play a role in processes related to learning and memory, sleep, thermoregulation, mood, motor activity, pain, sexual and aggressive behaviors, appetite, neurodegenerative regulation, and biological rhythms. Not surprisingly, serotonin is linked to pathophysiological conditions such as anxiety, depression, obsessive-compulsive disorders, schizophrenia, suicide, autism, migraine, emesis, alcoholism, and neurodegenerative disorders. With respect to anti-psychotic treatment approaches focused on the serotonin receptors, these types of therapeutics can generally be divided into two classes, the “typical” and the “a typical.” Both have anti-psychotic effects, but the “typicals” also include concomitant motor-related side effects (extra pyramidal syndromes, e.g., lip-smacking, tongue darting, locomotor movement, etc). Such side effects are thought to be associated with the compounds interacting with other receptors, such as the human dopamine D2 receptor in the nigro-striatal pathway. Haloperidol is considered a typical anti-psychotic, and Clozapine is considered an a typical anti-psychotic.

### SUMMARY OF THE INVENTION

In some embodiments, the present invention provides compositions comprising a neuroleptic and a modulator of a 5-HT2A receptor. In some embodiments of the compositions of the invention, the neuroleptic is selected from the group consisting of Haloperidol, Haloperidol decanoate, Clozapine, Benperidol; Chlorpromazine, Droperidol, Flupenthixol, Flupenthixol decanoate, Fluspiriline, Methotrimeprazine, Levomepromazine, Olanzapine, Oxypertine, Pericyazine, Perphenazine, Pimozide, Pipothiazine decanoate, Prochlorperazine, Promazine, Quetiapine, Remoxipride, Risperidone, Sertindole, Sulpiride, Thioridazine, Trifluoperazine, Zucopenthixol decanoate, Zuclopenthixol, and Clopixol, preferably Haloperidol. In further embodiments of the compositions of the invention, the modulator of the 5-HT2A receptor is an inverse agonist of the 5-HT2A receptor. In some embodiments of the compositions of the invention, the inverse agonist of the 5-HT2A receptor is N-[3-(4-bromo-2-methylpyrazol-3-yl)phenyl][(4-chlorophenyl)amino]carboxamide, or a derivative thereof.

In some embodiments of the compositions of the invention, the inverse agonist of the 5-HT2A receptor is N-[3-(4-bromo-2-methylpyrazol-3-yl)phenyl][(4-chlorophenyl)amino]carboxamide, or a derivative thereof, and the neuroleptic is Haloperidol.

In some embodiments of the invention, compositions are provided comprising a neuroleptic and a compound having the formula A:

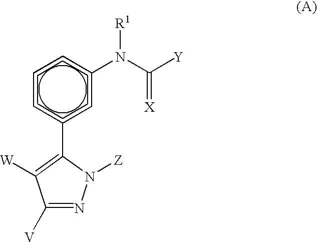

wherein:

W is lower alkyl (C₁₋₆), or halogen;

V is lower alkyl (C₁₋₆), H, or halogen;

X is either Oxygen or Sulfur;

Y is NR²R³, or (CH₂)ₘR⁴, or O(CH₂)ₙR⁴;

Z is lower alkyl (C₁₋₆);

m=0-4

n=0-4

R¹ is H or lower alkyl(C₁₋₄);

R² is H or lower alkyl(C₁₄);

R³ and R⁴ are independently a C₁₋₆ alkyl, or C₂₋₆ alkenyl, or cycloalkyl, or aryl group and each said group may be optionally substituted by up to four substituents in any position independently selected from CF₃, CCl₃, Me, NO₂, OH, OMe, OEt, CONR⁵R⁶, NRR⁶, OCF₃, SMe, COOR⁷, SO₂NR⁵R⁶, S₃, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl;

R⁵ and R⁶ are independently a H, or C₁₋₆ alkyl, or C₂₋₆ alkenyl, or cycloalkyl, or aryl, or CH₂ aryl group and each said group may be optionally substituted by up to four substituents in any position independently selected from CF₃, CCl₃, Me, NO₂, OH, OMe, OEt, CONR⁷R⁸, NR⁷R⁸, NHCOCH₃, OCF₃, SMe, COOR⁹, SO₃R⁷, SO₂NR⁷R⁸, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl wherein each of the C₃₋₆ cycloalkyl, C₁₋₆ alkyl, or aryl groups may be further optionally substituted by up to four substituents in any position independently selected from CF₃, CCl₃, Me, NO₂, OH, OMe, OEt, CONR⁸R⁹, NR⁸R⁹, NHCOCH₃, OCF₃, SMe, COOR⁷, SO₂NR⁸R⁹, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl, or R⁵ and R⁶ may form part of a 5, 6 or 7 membered cyclic structure which may be either saturated or unsaturated and that may contain up to four heteroatoms selected from O, N or S and said cyclic structure may be optionally substituted by up to four substituents in any position independently selected from CF₃, CCl₃, Me, NO₂, OH, OMe, OEt, OCF₃, SMe, COOR⁷, SO₂NR⁸R⁹, SO₃R⁷, NHCOCH₃, COEt, COMe, or halogen;

R⁷ may be independently selected from H or C₁₋₆ alkyl;

R⁸ and R⁹ are independently a H, or C₁₋₆ alkyl, or C₂₋₆ alkenyl, or cycloalkyl, or aryl, or CH₂ aryl group and each said group may be optionally substituted by up to four substituents in any position independently selected from halogen, CF₃, OCF₃, OEt, CCl₃, Me, NO₂, OH, OMe, SMe, COMe, CN, COOR⁷, SO₃R⁷, COEt, NHCOCH₃, or aryl;

an aryl moiety can be a 5 or 6 membered aromatic heterocyclic ring (containing up to 4 hetero atoms independently selected from N, 0, or S) or a 6 membered aromatic non-heterocyclic ring or a polycycle;

C₁₋₆ alkyl moieties can be straight chain or branched;

optionally substituted C₁₋₆ alkyl moieties can be straight chain or branched;

C₂₋₆ alkenyl moieties can be straight chain or branched; and

optionally substituted C₂₋₆ alkenyl moieties can be straight chain or branched, or a pharmaceutically acceptable acid addition salt thereof.

In some such embodiments, the compound of formula A has the formula:

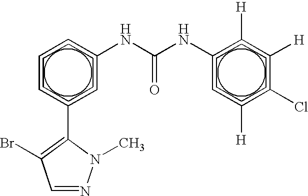

In further such embodiments, the neuroleptic is selected from the group consisting of Haloperidol; Haloperidol decanoate; Clozapine; Benperidol; Chlorpromazine; Droperidol; Flupenthixol; Flupenthixol decanoate; Fluspiriline; Methotrimeprazine; Levomepromazine; Olanzapine; Oxypertine; Pericyazine; Perphenazine; Pimozide; Pipothiazine decanoate; Prochlorperazine; Promazine; Quetiapine; Remoxipride; Risperidone; Sertindole; Sulpiride; Thioridazine; Trifluoperazine; Zucopenthixol decanoate; Zuclopenthixol; Clopixol, or combinations or subcombinations thereof, preferably wherein the neuroleptic is Haloperidol.

In some embodiments, the present invention provides method of reducing hyperlocomotor activity, methods of reducing stereotypy, methods of treating psychoses in a mammal while minimizing motor-related side effects, and methods of treating psychoses in a subject while minimizing extrapyramidal motor syndrome, said methods comprising the step of administering to a subject a pharmaceutically effective amount of a composition comprising a neuroleptic and a modulator of a 5-HT2A receptor.

In some embodiments of the methods of the invention, the modulator of the 5HT-2A receptor is a compound of formula A as described above, which in some preferred embodiments has the formula:

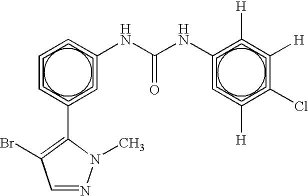

In further embodiments of the methods of the invention, the neuroleptic is selected from the group consisting of Haloperidol, Haloperidol decanoate, Clozapine, Benperidol; Chlorpromazine, Droperidol, Flupenthixol, Flupenthixol decanoate, Fluspiriline, Methotrimeprazine, Levomepromazine, Olanzapine, Oxypertine, Pericyazine, Perphenazine, Pimozide, Pipothiazine decanoate, Prochlorperazine, Promazine, Quetiapine, Remoxipride, Risperidone, Sertindole, Sulpiride, Thioridazine, Trifluoperazine, Zucopenthixol decanoate, Zuclopenthixol, and Clopixol, or combinations or subcombinations thereof, preferably wherein the neuroleptic is Haloperidol.

In some preferred embodiments of the methods of the invention, the neuroleptic is Haloperidol, and the modulator of the 5HT-2A receptor is a compound of formula A, preferably wherein the compound has the formula:

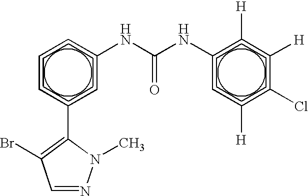

In some embodiments of the methods of the invention, subjects are identified as being in need of treatment of a psychosis susceptible to undesired side effects prior to administering to the subject the pharmaceutically effective amount of the composition of the invention.

### BRIEF DESCRIPTION OF THE DRAWINGS

In the following figures, bold typeface indicates the location of the mutation in the non-endogenous, constitutively activated receptor relative to the corresponding endogenous receptor.

FIG. 1 provides a graphic summary of the effect of co-administration of effective doses of AR116081 (20 mg/kg) and Haloperidol (0.05 mg/kg) on MK-801-induced hyperactivity in rats. Results are presented as total activity counts over the 180 min of exposure to the locomotor activity apparatus after administration of MK-801. Data (mean ±SEM) were analyzed by unpaired t-test (n—6-8/group).

FIG. 2 provides a graphic summary of time course of effect of co-administration of AR116081 and Haloperidol on MK-801-induced hyperactivity in rats. (S.E.M.s and statistical analysis are not shown for ease of data presentation).

### DETAILED DESCRIPTION

The present invention relates to the discovery that the beneficial effects of a neuroleptic in the treatment of psychoses, including schizophrenia, may be enhanced while decreasing the neurological side effects associated with such neuroleptics by administering, prior to, following, or concomitant with the neuroleptic, an effective amount of a modulator of 5-HT2A receptor, preferably an inverse agonist of the 5-HT2A receptor.

Schizophrenia is a psychopathic disorder of unknown origin, which usually appears for the first time in early adulthood and is marked by a number of characteristics, psychotic symptoms, progression, phasic development and deterioration in social behavior and professional capability in the region below the highest level ever attained. Characteristic psychotic symptoms are disorders of thought content (multiple, fragmentary, incoherent, implausible or simply delusional contents or ideas of persecution) and of mentality (loss of association, flight of imagination, incoherence up to incomprehensibility), as well as disorders of perceptibility (hallucinations), of emotions (superficial or inadequate emotions), of self-perception, of intentions and impulses, of interhuman relationships, and finally psychomotoric disorders (such as catatonia). Other symptoms are also associated with this disorder. (See, American Statistical and Diagnostic Handbook).

Haloperidol, described in U.S. Pat. No. 3,438,991, incorporated herein by reference, is a well-known neuroleptic agent used for treating psychoses, such as schizophrenia. A significant problem of currently marketed typical antipsychotics such as Haloperidol is the occurrence of extrapyramidal side effects, which usually occurs after antipsychotic therapy has begun. The extrapyramidal motor syndrome (EPS) is characterized by Parkinson-like symptoms resulting from the blockade of brain striatal doparnine D2 and Dl receptors (Snyder, S H. Am. J. Psychiatry, 138:461-468, 1981). The propensity of a potential therapeutic to block striatal dopamine receptors can be evaluated by measurement of the induction of catalepsy in rodents (Hoffman and Donavan, Psychopharmacology 120:128-133, 1995). Catalepsy is characterized by body rigidity and is commonly measured using the bar test in rats (Prinssen et al., Psychopharmacology, 144:20-29,1999).

Use of Haloperidol causes side effects including acute neuroleptic malignant syndrome (NMS) which has been reported in association with antipsychotic drugs as well as the more chronic dystonic syndrome known as which sometimes emerges during long-term antipsychotic use. (Physician's Desk Reference, pg. 2155 54^{th} Edition (2000)).

In some embodiments the present invention provides compositions comprising a neuroleptic and a modulator of a 5-HT2A receptor. In some embodiments the neuroleptic is selected from the group consisting of Haloperidol, Haloperidol decanoate, Clozapine, Benperidol; Chlorpromazine, Droperidol, Flupenthixol, Flupenthixol decanoate, Fluspiriline, Methotrimeprazine, Levomepromazine, Olanzapine, Oxypertine, Pericyazine, Perphenazine, Pimozide, Pipothiazine decanoate, Prochlorperazine, Promazine, Quetiapine, Remoxipride, Risperidone, Sertindole, Sulpiride, Thioridazine, Trifluoperazine, Zucopenthixol decanoate, Zuclopenthixol, and Clopixol, or combinations or subcombinations thereof. In some embodiments, the neuroleptic is an “a typical” neuroleptic. In some preferred embodiments, the neuroleptic is a “typical” neuroleptic. In some preferred embodiments the neuroleptic is Haloperidol.

As used herein in connection with the present invention, the term COMPOSITION refers to a combination having at least two components, for example a neuroleptic and a modulator of the 5-HT2A receptor. For example, and not limitation, a Pharmaceutical Composition is a Composition. The term COMPOSITION includes scenarios where the two or more compounds or components are present in the subject at the same time. For example, a composition has been administered to a subject when the two or more compounds identified as being part of such composition, or two or more components of a composition, are administered simultaneously, for example in a single dosage form such as a tablet or injection, or separately, as described below.

As used herein, the terms ADMINISTER or ADMINISTERING refer to the delivery of a COMPOSITION to a subject. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Methods and modes of administration are further described below. In the context of the present invention, a composition has been administered to a subject when the constituent compounds and/or components of the composition are introduced into a subject by whatever means such that the compounds and/or or components of the composition are present in the subject in effective amount at the same time. Thus, constituent compounds or components of a composition of the invention may be administered in the same manner or form, or in different manners or forms, including but not limited to the modalities described herein. As a non-limiting example, one compound or component may be delivered via a transdermal patch while the second compound or component may be delivered via an intravenous injection. Further, the two compounds or components may both be administered in the same manner, e.g., both compounds or components may be delivered as tablets. Further, the compounds or components may be administered in the same dosage form, e.g. all of the compounds or components being present in a single dosage form, for example a tablet.

As used herein, the terms CONTACT or CONTACTING refer to bringing at least two moieties together, whether in an in vitro system or an in vivo system.

As used herein, the term PHARMACEUTICAL COMPOSITION refers to a COMPOSITION comprising an effective amount of two or more compounds or components. As will be described in greater detail below, those of ordinary skill in the art will understand and appreciate the techniques appropriate for determining effective amounts of compounds or components in a COMPOSITION.

As used herein, the term NEUROLEPTIC refers to a class of medicaments for the treatment of mental disorders, especially psychoses. Examples of NEUROPLEPTCIS are well known to those of skill in the art.

As used herein, ABOUT is intended to refer to plus or minus 10%.

As used herein, the term UNDESIRED SIDE EFFECTS is intended to refer to side effects, both known as well as yet unknown, associated with the use of neuroleptics. Known side effects associated with the use of neurolpetics include, but are not limited to, acute neuroleptic malignant syndrome, concomitant motor-related side effects including extra pyramidal syndromes, e.g., lip-smacking, tongue darting, locomotor movement, and tardive dyskinesia; as well as other nonmotor-related side effects. UNDESIRED SIDE EFFECTS also include side effects that, although not serious enough to warrant medical attention, are factors in a subject's decision whether or not to use the compositions of the present invention. Such side effects include, but are not limited to, fatigue, insomnia, loss of appetite, nausea, localized or general skin irritation, irritability, headache, blurred vision, cramps, general malaise, and the like.

As used herein, the terms MODULATE and MODULATOR refer to compositions and the effects of such compositions on the 5-HT2A receptor. MODULATORS of the 5-HT2A receptor are compositions which affect the functionality of the receptor. Specifically, MODULATORS of the 5-HT2A receptor increase or decrease the functionality of the 5-HT2A receptor. In some preferred embodiments, MODULATORS increase or decease the functionality of the 5-HT2A receptor by at least 10%, at least 25%, at least 35%, at least 40%, at least 50%, at least 60%, at least 75%, at least 80%, at least 90%, at least 95%, and at least 100%, as compared to the functionality of the 5-HT2A receptor absent the composition. Examples of MODULATORS of the 5-HT2A receptor include agonists, inverse agonists, and antagonists of the 5-HT2A receptor.

AGONISTS shall mean compounds that activate the intracellular response when they bind to a receptor, or enhance GTP binding to membranes.

ANTAGONIST shall mean compounds that competitively bind to a receptor at the same site as agonists but which do not activate the intracellular response initiated by the active form of the receptor, and can thereby inhibit the intracellular responses by agonists or partial agonists. ANTAGONISTS do not diminish the baseline intracellular response in the absence of an agonist or partial agonist.

INVERSE AGONISTS shall mean compounds which bind to either the endogenous form of a receptor or to the constitutively activated form of the receptor, and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50%, and most preferably by at least 75%, as compared with the baseline response in the absence of the inverse agonist.

As used herein, the term SUBJECT refers to the animal to which the compositions of the present invention are administered or the target of the methods of the present invention. Suitable SUBJECTS include vertebrates, preferably mammals, and most preferably humans.

In some embodiments the compositions of the present invention comprise an inverse agonist of the 5-HT2A receptor and a neuroleptic. In some preferred embodiments, the inverse agonist of the 5-HT2A receptor is N-[3-(4-bromo-2-methylpyrazol-3-yl)phenyl][(4-chlorophenyl)amino]carboxamide, or a derivative thereof.

As used herein, the term “derivative” refers to addition salts, chelates, complexes and the like as are known in the art, preferably wherein they are pharmaceutically acceptable.

The compounds of the invention may contain amino groups and, therefore, are capable of forming salts with various inorganic and organic acids. Such salts are also within the scope of this invention. Representative salts include inorganic addition salts such as phosphate, hydrochloride, hydrobromide, hydroiodide, hemisulfate, sulfate, bisulfate and nitrate, and organic salts including, for example, acetate, benzoate, butyrate, citrate, fumarate, heptanoate, hexanoate, lactate, maleate, succinate and tartrate. The salts can be formed by conventional means, such as by reacting the free base form of the product with one or more equivalents of the appropriate acid in a solvent or medium in which the salt is insoluble, or in a solvent such as water which is later removed in vacuo or by freeze drying. The salts also can be formed by exchanging the anions of an existing salt for another anion on a suitable ion exchange resin.

Suitable salts of the compounds of the present invention are pharmaceutically acceptable conventional non-toxic salts and can be an organic acid addition salt (e.g. formate, acetate, trifluoroacetate, maleate, tartarate, oxalate, methanesulfonate, benzenesulfonate, toluenesulfonate, etc.), an inorganic acid addition salt (e.g. hydrochloride, hydrobromide, sulfate, phosphate, etc.), a salt with an amino acid (e.g. aspartic acid salt, glutamic acid salt, etc.), or the like.

Examples of the salts of the compound (I) include a pharmaceutically acceptable salt, etc. such as an acid addition salt (e.g. a salt with acetic acid, lactic acid, succinic acid, maleic acid, tartaric acid, citric acid, gluconic acid, ascorbic acid, benzoic acid, methanesulfonic acid, p-toluenesulfonic acid, cinnamic acid, fumaric acid, phosphoric acid, hydrochloric acid, nitric acid, hydrobromic acid, hydriodic atom acid, sulfamic acid, sulfuric acid, etc.), a metal salt (e.g. a salt with sodium, potassium, magnesium, calcium, etc.), an organic base (e.g. trimethylamine, triethylamine, pyridine, picoline, N-methylpyrrolidine, N-methylpiperidine, N-methylmolpholine, etc.), etc.

Examples of the acid halide include acid chloride, acid bromide, etc. Examples of the mixed acid anhydride include mono-C₁₋₄ alkyl carbonate mixed acid anhydride (e.g. a mixed acid anhydride of a free acid (V) with monomethylcarbonate, monoethylcarbonate, monoisopropylcarbonate, monoisobutylcarbonate, mono-(tert-butyl)carbonate, mono-benzylcarbonate, mono(p-nitrobenzyl)carbonate, monoallylcarbonate, etc.), C₁₋₆ alicyclic carboxylic acid mixed acid anhydride (e.g. a mixed acid anhydride of a free acid (V) with acetic acid, cyanoacetic acid, propionic acid, butyric acid, isobutyric acid, valeric acid, isovaleric acid, pivalic acid, trifluoroacetic acid, trichloroacetic acid, acetoacetic acid, etc.), C₇₋₁₁ aromatic carboxylic acid mixed acid anhydride (e.g. a mixed acid anhydride of a free acid (V) with benzoic acid, p-toluic acid, p-chlorobenzoic acid, etc.), organic sulfonic acid mixed acid anhydride (e.g. a mixed acid anhydride with methanesulfonic acid, ethanesulfonic acid, benzenesulfonic acid, p-toluenesulfonic acid, etc.), etc. Examples of the active amide include an amide with nitrogen-containing heterocyclic compound (e.g. an acid amide of a free acid (V) with pyrazole, imidazole, benzotriazole, etc.; said nitrogen-containing heterocyclic compound is optionally substituted with C₁₋₆ alkyl (e.g. methyl, ethyl, propyl, isopropyl, butyl, isobutyl, sec-butyl, tert-butyl, etc.), C₁₋₆ alkoxy (e.g. methoxy, ethoxy, propoxy, isopropoxy, butoxy, tert-butoxy, etc.), a halogen atom (e.g. fluorine, chlorine, bromine, etc.), oxo, thioxo, C₁₋₆ alkylthio (e.g. methylthio, ethylthio, propylthio, butylthio, etc.

Examples of the active ester include organic phosphoric acid ester (e.g. diethoxyphosphoric acid ester, diphenoxyphosphoric acid ester, etc.), p-nitrophenylester, 2,4-dinitrophenylester, cyanomethylester, pentachlorophenylester, N-hydroxysuccinimide ester, N-hydroxy-phthalimide ester, 1-hydroxybenzotriazole ester, 6-chloro-1-hydroxy-benzotriazole ester, 1-hydroxy-1H-2-pyridone ester, etc.

Examples of the active thioester include ester with aromatic heterocyclic thiol compound (e.g. 2-pyridylthiol ester, 2-benzo-thiazolylthiol ester), etc., said heterocyclic group being optionally substituted with C₁₋₆ alkyl (e.g. methyl, ethyl, propyl, isopropyl, butyl, isobutyl, sec-butyl, tert-butyl, etc.), C₁₋₆ alkoxy (e.g. methoxy, ethoxy, propoxy, isopropoxy, butoxy, tert-butoxy, etc.), a halogen atom (e.g. fluorine, chlorine, bromine, etc.), C₁₋₆ alkyl thio (e.g. methylthio, ethylthio, propylthio, butylthio, etc.), etc.

The present invention also encompasses the pharmaceutically acceptable esters, amides, complexes, chelates, hydrates, crystalline or amorphous forms, metabolites, metabolic precursors or prodrugs of the compounds of formula A. Pharmaceutically esters and amides can be prepared by reacting, respectively, a hydroxy or amino functional group with a pharmaceutically acceptable organic acid, such as identified below. A prodrug is a drug which has been chemically modified and may be biologically inactive at its site of action, but which is degraded or modified by one or more enzymatic or other in vivo processes to the parent bioactive form. Generally, a prodrug has a different pharmacokinetic profile than the parent drug such that, for example, it is more easily absorbed across the mucosal epithelium, it has better salt formation or solubility and/or it has better systemic stability (e.g., an increased plasma half-life).

The compounds of the present invention can be used in their neat form or in the form of pharmaceutically-acceptable salts derived from inorganic or organic acids. Examples of acids which may be employed to form pharmaceutically acceptable acid addition salts of compounds of the present invention include such inorganic acids as hydrochloric acid, sulphuric acid and phosphoric acid and such organic acids as oxalic acid, maleic acid, succinic acid and citric acid. These salts thus include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hernisulfate, heptanoate, hexanoate, fumarate, hydrochloride, hydrobromide hydroiodide, 2-hydroxy-ethanesulfonate, lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, p-toluenesulfonate and undecanoate.

Also, the basic nitrogen-containing groups can be quatemized with such agents as lower alkyl halides, such as methyl, ethyl, propyl, and butyl chlorides, bromides and iodides; dialkyl sulfates, like dimethyl, diethyl, dibutyl and diamyl sulfates, long chain halides such as decyl, lauryl, myristyl and stearyl chlorides, omides and iodides, aralkyl halides like benzyl and phenethyl bromides and others. Water or oil soluble or dispersible products are thereby generally obtained.

The pharmaceutically acceptable salts of the compounds of the present invention also can exist as various solvates, such as with water, methanol, ethanol, dimethylformamide, ethyl acetate and the like. Mixtures of such solvates also can be prepared. Such solvates are within the scope of the present invention.

In some more preferred embodiments, the inverse agonist of the 5-HT2A receptor is a compound of formula A having the formula:

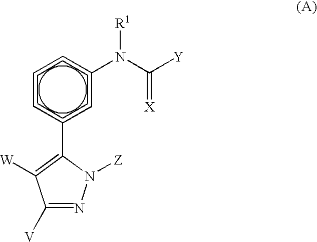

wherein:

W is lower alkyl (C₁₋₆), or halogen;

V is lower alkyl (C₁₋₆), H, or halogen;

X is either Oxygen or Sulfur;

Y is NR²R³, or (CH₂)ₘR⁴, or O(CH₂)ₙR⁴;

Z is lower alkyl (C₁₋₆);

m=0-4

n=0-4

R¹ is H or lower alkyl(C₁₋₄);

R² is H or lower alkyl(C₁₋₄);

R³ and R⁴ are independently a C₁₋₆ alkyl, or C₂₋₆ alkenyl, or cycloalkyl, or aryl group and each said group may be optionally substituted by up to four substituents in any position independently selected from CF₃, CCl₃, Me, NO₂, OH, OMe, OEt, CONR⁵R⁶, NR⁵R⁶, OCF₃, SMe, COOR⁷, SO₂NR⁵R⁶, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl;

R⁵ and R⁶ are independently a H, or C₁₋₆ alkyl, or C₂₋₆ alkenyl, or cycloalkyl, or aryl, or CH₂ aryl group and each said group may be optionally substituted by up to four substituents in any position independently selected from CF₃, CCl₃, Me, NO₂, OH, OMe, OEt, CONR⁷R⁸, NR⁷R⁸, NHCOCH₃, OCF₃, SMe, COOR⁹, SO₃R⁷, SO₂NR⁷R⁸, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl wherein each of the C₃₋₆ cycloalkyl, C₁₋₆ alkyl, or aryl groups may be further optionally substituted by up to four substituents in any position independently selected from CF₃, CCl₃, Me, NO₂, OH, OMe, OEt, CONR⁵R⁹, NR⁸R⁹, NHCOCH₃, OCF₃, SMe, COOR⁷, SO₂NR⁸R⁹, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl,

or R⁵ and R⁶ may form part of a 5, 6 or 7 membered cyclic structure which may be either saturated or unsaturated and that may contain up to four heteroatoms selected from O, N or S and said cyclic structure may be optionally substituted by up to four substituents in any position independently selected from CF₃, CCl₃, Me, NO₂, OH, OMe, OEt, OCF₃, SMe, COOR⁷, SO₂NR⁸R⁹, SO₃R⁷, NHCOCH₃, COEt, COMe, or halogen;

R⁷ may be independently selected from H or C₁₋₆ alkyl;

R⁸ and R⁹ are independently a H, or C₁₋₆ alkyl, or C₂₋₆ alkenyl, or cycloalkyl, or aryl, or CH₂ aryl group and each said group may be optionally substituted by up to four substituents in any position independently selected from halogen, CF₃, OCF₃, OEt, CCl₃, Me, NO₂, OH, OMe, SMe, COMe, CN, COOR⁷, SO₃R⁷, COEt, NHCOCH₃, or aryl;

an aryl moiety can be a 5 or 6 membered aromatic heterocyclic ring (containing up to 4 hetero atoms independently selected from N, O, or S) or a 6 membered aromatic non-heterocyclic ring or a polycycle;

C₁₋₆ alkyl moieties can be straight chain or branched;

optionally substituted C₁₋₆ alkyl moieties can be straight chain or branched;

C₂₋₆ alkenyl moieties can be straight chain or branched; and

optionally substituted C₂₋₆ alkenyl moieties can be straight chain or branched, or a pharmaceutically acceptable acid addition salt thereof.

In some preferred embodiments, the inverse agonist of the 5-HT2A receptor is a compound having the formula:

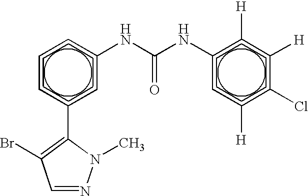

In some embodiments the invention provides methods of reducing hyperlocomotor activity comprising administering to a subject a pharmaceutically effective amount of a composition of the present invention.

In some embodiments the invention provides methods of reducing stereotypy comprising administering to a subject a pharmaceutically effective amount of a composition of the present invention.

In other embodiments the invention provides methods of treating psychoses in a subject while minimizing motor-related side effects comprising administering to a subject a pharmaceutically effective amount of a composition of the present invention.

In some embodiments the invention provides methods of treating psychoses in a subject while minimizing extrapyramidal motor syndrome comprising administering to a subject a pharmaceutically effective amount of a composition of the present invention.

In some embodiments, the methods recited above further comprise the step of identifying a subject, the subject susceptible to undesired side effects of neuroleptic therapy, prior to administration of the composition of the present invention. Those skilled in the art are credited with the ability to identify a subject susceptible to undesired side effects of neuroleptic therapy.

A preferred compound falling within the scope of general Formula (A) is referred to as compound “AR116081”, and has the following structure:

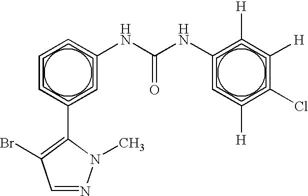

AR116081 is a selective inverse agonist of the serotonin receptor, 5-HT2A, and has antipsychotic properties.

Compounds of formula A in combination with neuroleptics such as Haloperidol can be used as a putative therapeutic for psychotic disorders in humans. In some embodiments the use of AR116081 in combination with Haloperidol is preferred Preferred compositions are those containing a therapeutically effective amount of a neuroleptic and a therapeutically effective amount of a modulator of the 5-HT2A receptor. In some preferred embodiments, a composition comprising AR116081, or a pharmaceutically acceptable acid addition salt thereof, is co-administered prior to, following, or concomitantly with Haloperidol in the same or a different therapeutically effective dosage form. As further described below, determination of the proper dosage for a particular situation is within the skill of the art. Generally, treatment is initiated with smaller dosages which are less than the optimum dose of the compound. Thereafter the dosage is increased by small increments until the optimum effect under the circumstances is reached. Further description of modes and materials for administration of the compositions of the present invention is provided below.

For moderate symptomatology, the initial dosage range of neuroleptics, including Haloperidol, for adults is generally from about 0.5 mg to 2.0 mg b.i.d. or t.i.d. and about 3.0 mg to 5.0 mg b.i.d. or t.i.d. for severe symptomatology. To achieve prompt control, higher doses may be required in some cases. For children between the ages of 3 and 12 years (weight range 15 to 40 kg) therapy should begin at the lowest dose possible (0.5 mg per day). Upon achieving a satisfactory therapeutic response, dosage should be gradually reduced to the lowest effective maintenance level. (See, Physicians Desk Reference, 54^{th} Edition (2000)). Further descriptions of dosing information are provided below.

The present invention further includes a method for treating psychoses, e.g., schizophrenia, in a subject suffering therefrom comprising administering to such subjects a pharmaceutical composition comprising a modulator of the 5-HT2A receptor, preferably AR116081, and a neuroleptic, preferably Haloperidol, in appropriate unit dosage form.

The present invention is further related to the treatment of psychoses, e.g., schizophrenia, by attenuating the effects of the antagonist MK-801 at the 5-HT2 receptor, preferably the attenuation of MK-801 is accomplished through the use of a composition comprising a modulator of the 5-HT2A receptor, preferably AR116081, and a neuroleptic, preferably Haloperidol.

A well-known animal model for detecting potential therapeutic agents in the treatment of schizophrenia can be created by inducing animals with MK-801, an antagonist of typical and a typical neuroleptics such as Haloperidol and Clozapine, respectively. Briefly, in rodents, the non-competitive N-methyl-D-aspartate (NMDA) glutamate receptor antagonists PCP and MK-801 produce a behavioral syndrome characterized by rapid locomotor activity and stereotypy. This behavioral activation has been determined to be a valid animal model of schizophrenia because the symptoms of the disease are directly or indirectly related to altered glutamate transmission at the NMDA receptor. (See, Hoffman, D. C., J. Neural. Transm. 89:1-10, 1992).

Pharmaceutical compositions for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent such as lipids, liposomes, fatty acids, fatty acid esters, steroids, chelating agents and surfactants.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which compositions of the invention are administered in conjunction with one or more penetration enhancers surfactants and chelators. Preferred surfactants include fatty acids and/or esters or salts thereof, bile acids and/or salts thereof. Prefered bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, and sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. Compositions of the invention may be delivered orally in granular form including sprayed dried particles, or complexed to form micro or nanoparticles.

Compositions for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The compositions of the present invention may conveniently be presented in unit dosage form and may be prepared according to conventional techniques well known in the pharmaceutical industry. Such techniques include the step of bringing into association the active ingredients with the pharmaceutical carrier(s) or excipient(s). In general the formulations are prepared by uniformly and intimately contacting the active ingredients with liquid carriers or finely divided solid carriers or both, and then, if necessary, shaping the product.

The compositions of the present invention may be formulated into any of many possible dosage forms including, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

The compositions of the present invention may also be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

The compositions of the present invention may be prepared and formulated as emulsions. Emulsions are typically heterogenous systems of one liquid dispersed in another in the form of droplets usually exceeding 0.1 μm in diameter. (Pharmaceutical Dosage Forms, Lieberman, Rieger and Banker (Eds.), 1988, Marcel Dekker, Inc., New York, N.Y., volume 1; Remington's Pharmaceutical Sciences, Mack Publishing Co., Easton, Pa., 1985). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug that may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that comprise more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Emulsifiers may broadly be classified into four categories: synthetic surfactants, naturally occurring emulsifiers, absorption bases, and finely dispersed solids (Pharmaceutical Dosage Forms, Lieberman, Rieger and Banker (Eds.), 1988, Marcel Dekker, Inc., New York, N.Y., volume 1). Synthetic surfactants, also known as surface active agents, may also be used.

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials may also included in emulsion formulations and contribute to the properties of emulsions. These include fats, oils, waxes, fatty acids, fatty alcohols, fatty esters, humectants, hydrophilic colloids, preservatives and antioxidants (Pharmaceutical Dosage Forms, Lieberman, Rieger and Banker (Eds.), 1988, Marcel Dekker, Inc., New York, N.Y., volume 1).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammomum salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been extensively reviewed (Pharmaceutical Dosage Forms, Lieberman, Rieger and Banker (Eds.), 1988, Marcel Dekker, Inc., New York, N.Y., volume 1). Emulsion formulations for oral delivery have been very widely used because of ease of formulation, efficacy from an absorption and bioavailability standpoint. (Pharmaceutical Dosage Forms, Lieberman, Rieger and Banker (Eds.), 1988, Marcel Dekker, Inc., New York, N.Y., volume 1). Mineral-oil base laxatives, oil-soluble vitamins and high fat nutritive preparations are among the materials that have commonly been administered orally as o/w emulsions.

In some embodiments, the compositions are formulated as microemulsions. A microemulsion may be defined as a system of water, oil and amphiphile which is a single optically isotropic and thermodynamically stable liquid solution (Pharmaceutical Dosage Forms, Lieberman, Rieger and Banker (Eds.), 1988, Marcel Dekker, Inc., New York, N.Y., volume 1). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Remington's Pharmaceutical Sciences, Mack Publishing Co., Easton, Pa., 1985, incorporated herein by reference).

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (P0310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al, J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in pharmaceutical applications. It is expected that the microemulsion compositions and formulations of the present invention will facilitate the increased systemic absorption of compositions from the gastrointestinal tract, as well as improve the local cellular uptake of compositions within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The compositions can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances that increase the viscosity of the suspension including, for example, sodium carboxymethylcellulose, sorbitol and/or dextran. The suspension may also contain stabilizers.

The formulation of the compositions of the present invention and their subsequent administration is within the skill of those in the art. Dosing is dependent on severity and responsiveness of the disease state to be treated, with the course of treatment lasting from several days to several months, or until a cure is effected or a diminution of the disease state or side effect is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can readily estimate repetition rates for dosing based on measured residence times and concentrations of the drug in bodily fluids or tissues. Following successful treatment, it may be desirable to have the patient undergo maintenance therapy to prevent the recurrence of the disease state, wherein the composition is administered in maintenance doses, ranging from 0.01 μg to 100 g per kg of body weight, once or more daily, to once every 20 years.

The following Example further illustrates the invention.

### EXAMPLE 1

Materials

The following materials were used in the examples provided below:

### AR116081

AR116081 and other compounds of Formula A useful in the present invention may be prepared as disclosed in U.S. Pat. No. 6,140,509, issued on Oct. 31, 2000; U.S. Pat. No. 6,107,324 issued on Aug. 22, 2000; U.S. Pat. No. 6,150,393 issued on Nov. 21, 2000; and pending, commonly-owned U.S. application Ser. Nos. 10/057,818 and 10/055,555, each of which is incorporated by reference in its entirety. AR116081 has the following structure:

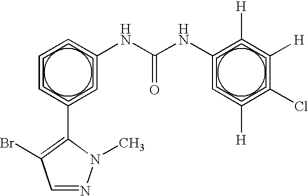

### Haloperidol

Haloperidol was purchased from RBI lot # TOW-1990 dissolved in distilled water and pH at 5-6, volume injection 1 cc/kg. MK-801.

MK801 hydrogen maleate at a dose of 0.3 mg/kg salt was purchased from RBI/Sigma Lot # 108H4705 dissolved in saline, volume injection icc/kg.

### Animals

Male Sprague-Dawley rats (200-325g) were purchased from Harlen Sprague (San Diego, Calif.).

In Vivo Analysis

Antagonism of MK801-Induced Hyperlocomotion: a Model of Potential Antipsychotic (“antipositive”) Activity

In rodents, the non-competitive NMDA receptor antagonist MK-801 induces significant increases in locomotor activity and stereotypy. Because part of the symptomatology of schizophrenia may be related to altered glutamate transmission at the NMDA receptor, the reversal of MK-801-induced hyperlocomotor activity in rodents has been used routinely as an animal model for detecting potential antipsychotic activity (Hoffinan, D. C., J. Neural. Transm. 89:1-10, 1992).

Motor function was assessed using automated locomotor activity cages (Hamilton-Kinder, San Diego) comprised of standard rodent cages surrounded by photocell beams, allowing for automated recording of motor activity. The animals were under no motivational constraints and were free to move around the cages. In this test, male Sprague-Dawley rats (200-325 g body weight) were food deprived overnight prior to testing. On the testing day, they were administered AR116081 orally at a dose of 10 or 20 mg/kg base (substance # 127190 suspended in 1% Tween 80 and 99% distilled water solution, volume injection 10 cc/kg) immediately followed by subcutaneous administration of Haloperidol at a dose of 0.05 mg/kg base (RBI lot # TOW-1990 dissolved in distilled water and pH at 5-6, volume injection 1 cc/kg). The treated rats were placed in the locomotor activity cages 30 min after co-administration of compounds for a 30 min habituation period to the cages and recording of basal locomotor activity. Animals then received (via subcutaneous administration) (+)-MK801 hydrogen maleate at a dose of 0.3 mg/kg salt (RBI/Sigma Lot #108H4705 dissolved in saline, volume injection 1 cc/kg). The rats were immediately placed back into the locomotor activity cages, and activity was measured for 180 min.

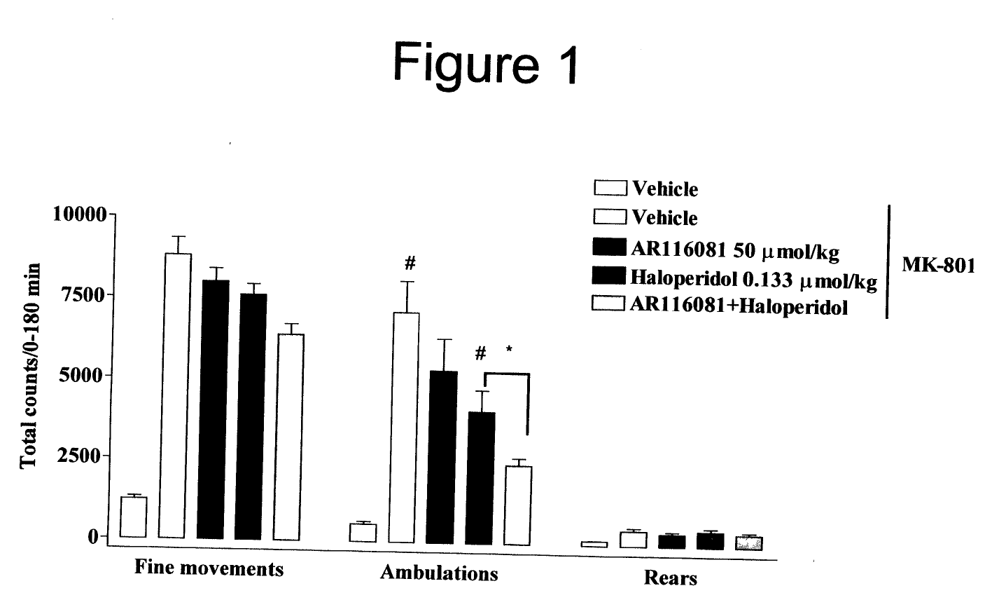

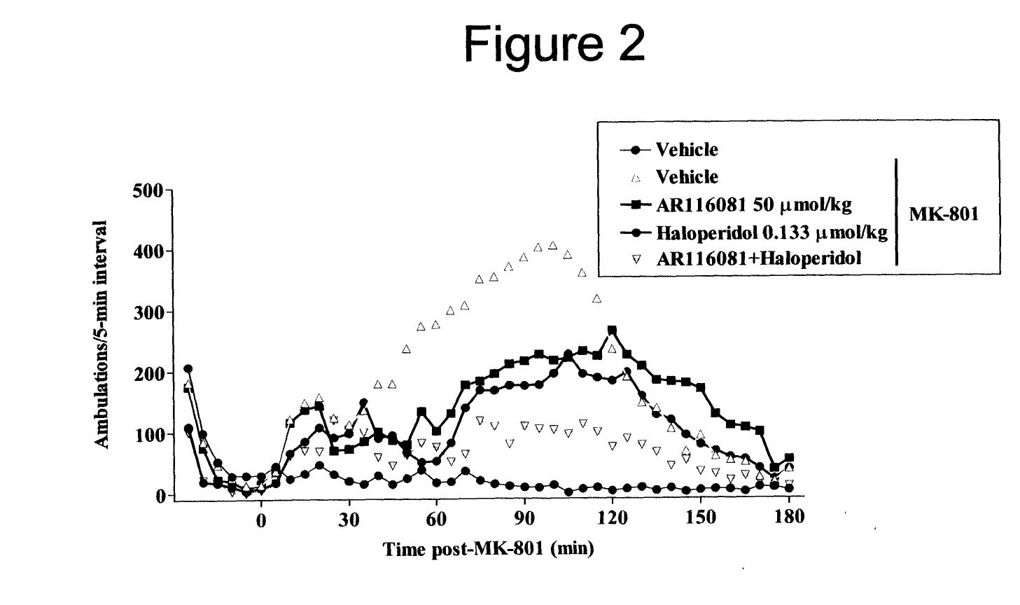

As shown in FIG. 1, MK-801 produced a significant increase of all three motor parameters recorded, e.g., ambulation (i.e., walking and running), fine movement of the body at rest (i.e., grooming, licking) and rearing activity (i.e., standing on hindlimbs). FIGS. 1 and 2 both evidence that AR116081 and Haloperidol attenuated MK-801-induced hyperactivity as measured by a decrease in ambulation over a time course of 180 minutes. In combination, Haloperidol and AR116081 significantly attenuated the effect of MK-801 by approximately 50% of the initial reversal effect induced by each compound alone. (See, FIGS. 1 and 2).

The same effect was observed on fine movements, a measure of stereotypies. When AR116081 or Haloperidol was administered individually, each attenuated but did not significantly reverse the effect of MK801. Combined, the two compounds produced a significant decrease of MK-801-induced hyperactivity. No effects were observed on rear measures. (See, FIG. 1).

These data suggest that AR116081 potentiates the effect of the neuroleptic Haloperidol in a model of psychosis in rats. The present invention evidence that in combination, modulators of the 5-HT2A receptor, preferably AR116081, and neuroleptics, preferably Haloperidol, preferably at a low dosage, will reverse the hyperactivity in the rat model, thereby potentially reducing the side effects usually associated with neuroleptics (e.g., extrapyramidal motor syndrome and tardive dyskinesia, etc.).

The reference works, patents, patent applications, and scientific literature, and other printed publications that are mentioned of referred to herein are hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A composition comprising a neuroleptic and a modulator of a 5-HT2A receptor.

2. The composition of claim 1 wherein the neuroleptic is selected from the group consisting of Haloperidol, Haloperidol decanoate, Clozapine, Benperidol; Chlorpromazine, Droperidol, Flupenthixol, Flupenthixol decanoate, Fluspiriline, Methotrimeprazine, Levomepromazine, Olanzapine, Oxypertine, Pericyazine, Perphenazine, Pimozide, Pipothiazine decanoate, Prochlorperazine, Promazine, Quetiapine, Remoxipride, Risperidone, Sertindole, Sulpiride, Thioridazine, Trifluoperazine, Zucopenthixol decanoate, Zuclopenthixol, and Clopixol.

3. The composition of claim 2 wherein the neuroleptic is Haloperidol.

4. The composition of claim 1 wherein the modulator of the 5-HT2A receptor is an inverse agonist of the 5-HT2A receptor.

5. The composition of claim 4 wherein the inverse agonist of the 5-HT2A receptor is N-[3-(4-bromo-2-methylpyrazol-3-yl)phenyl][(4-chlorophenyl)amino]carboxamide, or a derivative thereof.

6. The composition of claim 5 wherein the neuroleptic is Haloperidol.

7. A composition comprising a neuroleptic and a compound having the formula A: 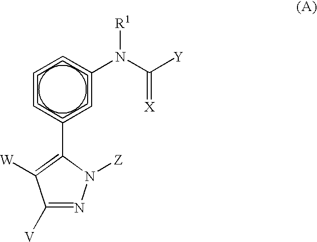
wherein:
W is lower alkyl (C₁₋₆), or halogen;
V is lower alkyl (C₁₋₆), H, or halogen;
X is either Oxygen or Sulfur;
Y is NR²R³, or (CH₂)ₘ⁴, or O(CH₂)ₙR⁴;
Z is lower alkyl (C₁₋₆);
m=0-4
m=0-4
R¹ is H or lower alkyl(C₁₋₄);
R² is H or lower alkyl(C₁₋₄);
R³ and R⁴ are independently a C₁₋₆ alkyl, or C₂₋₆ alkenyl, or cycloalkyl, or aryl group and each said group may be optionally substituted by up to four substituents in any position independently selected from CF₃, CCl₃, Me, NO₂, OH, OMe, OEt, CONR⁵R6, NR⁵R⁶, OCF₃, SMe, COOR7, SO₂NR⁵R⁶, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl;
R⁵ and R⁶ are independently a H, or C₁₋₆ alkyl, or C₂₋₆ alkenyl, or cycloalkyl, or aryl, or CH₂ aryl group and each said group may be optionally substituted by up to four substituents in any position independently selected from CF₃, CCl₃, Me, NO₂, OH, OMe, OEt, CONR⁷R⁸, NR⁷R⁸, NHCOCH₃, OCF₃, SMe, COOR⁹, SO₃R⁷, SO₂NR⁷R⁸, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl wherein each of the C₃₋₆ cycloalkyl, C₁₋₆ alkyl, or aryl groups may be further optionally substituted by up to four substituents in any position independently selected from CF₃, CCl₃, Me, NO₂, OH, OMe, OEt, CONR⁸R^{p}, NR⁸R⁹, NHCOCH₃, OCF₃, SMe, COOR⁷, SO₂NR⁸R⁹, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl,
or R⁵ and R⁶ may form part of a 5, 6 or 7 membered cyclic structure which may be either saturated or unsaturated and that may contain up to four heteroatoms selected from O N or S and said cyclic structure may be optionally substituted by up to four substituents in any position independently selected from CF₃, CCl₃, Me, NO₂, OH, OMe, OEt, OCF₃, SMe, COOR⁷, SO₂NR⁸R⁹, SO₃R⁷, NHCOCH₃, COEt, COMe, or halogen;
R⁷ may be independently selected from H or C₁₋₆ alkyl;
R⁸ and R⁹ are independently a H, or C₁₋₆ alkyl, or C₂₋₆ alkenyl, or cycloalkyl, or aryl, or CH₂ aryl group and each said group may be optionally substituted by up to four substituents in any position independently selected from halogen, CF₃, OCF₃, OEt, CCl₃, Me, NO₂, OH, OMe, SMe, COMe, CN, COOR⁷, SO₃R⁷, COEt, NHCOCH₃, or aryl;
an aryl moiety can be a 5 or 6 membered aromatic heterocyclic ring (containing up to 4 hetero atoms independently selected from N, O, or S) or a 6 membered aromatic non-heterocyclic ring or a polycycle;
C₁₋₆ alkyl moieties can be straight chain or branched;
optionally substituted C₁₋₆ alkyl moieties can be straight chain or branched;
C₂₋₆ alkenyl moieties can be straight chain or branched; and
optionally substituted C₂₋₆ alkenyl moieties can be straight chain or branched, or a pharmaceutically acceptable acid addition salt thereof.

8. The composition of claim 6 wherein the compound of formula A has the formula:

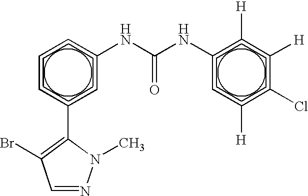

9. The composition of claim 8 wherein the neuroleptic is selected from the group consisting of Haloperidol, Haloperidol decanoate, Clozapine, Benperidol; Chlorpromazine, Droperidol, Flupenthixol, Flupenthixol decanoate, Fluspiriline, Methotrimeprazine, Levomepromazine, Olanzapine, Oxypertine, Pericyazine, Perphenazine, Pimozide, Pipothiazine decanoate, Prochlorperazine, Promazine, Quetiapine, Remoxipride, Risperidone, Sertindole, Sulpiride, Thioridazine, Trifluoperazine, Zucopenthixol decanoate, Zuclopenthixol, and Clopixol.

10. The composition of claim 9 wherein the neuroleptic is Haloperidol.

11. A method of reducing hyperlocomotor activity comprising administering to a subject a pharmaceutically effective amount of the composition of claim 1.

12. The method of claim 11 wherein the modulator of the 5HT-2A receptor is a compound of formula A having the formula: 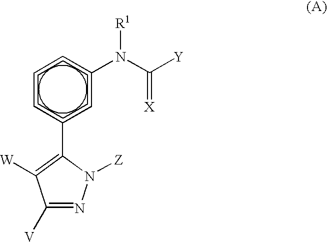
wherein:
W is lower alkyl (C₁₋₆), or halogen;
V is lower alkyl (C₁₋₆), H, or halogen;
X is either Oxygen or Sulfur;
Y is NR²R³, or (CH₂)ₘR⁴, or O(CH₂)ₙR⁴;
Z is lower alkyl (C₁₋₆);
m=0-4
n=0-4
R¹ is H or lower alkyl(C₁₋₄);
R² is H or lower alkyl(C₁₋₄);
R³ and R⁴ are independently a C₁₋₆ alkyl, or C₂₋₆ alkenyl, or cycloalkyl, or aryl group and each said group may be optionally substituted by up to four substituents in any position independently selected from CF₃, CCl₃, Me, NO₂, OH, OMe, OEt, CONR⁵R⁶, NR⁵R⁶, OCF₃, SMe, COOR⁷, SO₂NR⁵R⁶, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl;
R⁵ and R⁶ are independently a H, or C₁₋₆ alkyl, or C₂₋₆ alkenyl, or cycloalkyl, or aryl, or CH₂ aryl group and each said group may be optionally substituted by up to four substituents in any position independently selected from CF₃, CCl₃, Me, NO₂, OH, OMe, OEt, CONR⁷R⁸, NR⁷R⁸, NHCOCH₃, OCF₃, SMe, COOR⁹, SO₃R⁷, SO₂NR⁷R⁸, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl wherein each of the C₃₋₆ cycloalkyl, C₁₋₆ alkyl, or aryl groups may be further optionally substituted by up to four substituents in any position independently selected from CF₃, CCl₃, Me, NO₂, OH, OMe, OEt, CONR⁸R⁹, NR⁸R⁹, NHCOCH₃, OCF₃, SMe, COOR⁷, SO₂NR⁸R⁹, SO₃R⁷, COMe, COEt, CO-lower alkyl, SCF₃, CN, C₂₋₆ alkenyl, H, halogens, C₁₄ alkoxy, C₃₋₆ cycloalkyl, C₁₋₆ alkyl, and aryl,
or R⁵ and R⁶ may form part of a 5, 6 or 7 membered cyclic structure which may be either saturated or unsaturated and that may contain up to four heteroatoms selected from O, N or S and said cyclic structure may be optionally substituted by up to four substituents in any position independently selected from CF₃, CCl₃, Me, NO₂, OH, OMe, OEt, OCF₃, SMe, COOR7, SO₂NR R , SO₃R⁷, NHCOCH₃, COEt, COMe, or halogen;
R⁷ may be independently selected from H or C₁₋₆ alkyl;
R⁸ and R⁹ are independently a H, or C₁₋₆ alkyl, or C₂₋₆ alkenyl, or cycloalkyl, or aryl, or CH₂ aryl group and each said group may be optionally substituted by up to four substituents in any position independently selected from halogen, CF₃, OCF₃, OEt, CCl₃, Me, NO₂, OH, OMe, SMe, COMe, CN, COOR⁷, SO₃R⁷, COEt, NHCOCH₃, or aryl;
an aryl moiety can be a 5 or 6 membered aromatic heterocyclic ring (containing up to 4 hetero atoms independently selected from N, O, or S) or a 6 membered aromatic non-heterocyclic ring or a polycycle;
C₁₋₆ alkyl moieties can be straight chain or branched;
optionally substituted C₁₋₆ alkyl moieties can be straight chain or branched;
C₂₋₆ alkenyl moieties can be straight chain or branched; and
optionally substituted C₂₋₆ alkenyl moieties can be straight chain or branched, or a pharmaceutically acceptable acid addition salt thereof.

13. The method of claim 11 wherein the neuroleptic is Haloperidol and the modulator of the 5HT-2A receptor has the formula:

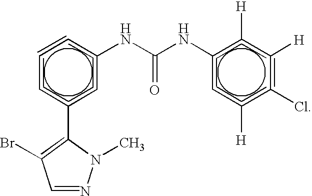

14. A method of reducing stereotypy comprising administering to a subject a pharmaceutically effective amount of the composition of claim 1.

15. The method of claim 14 wherein the modulator of the 5HT-2A receptor is a compound of formula A having the formula: 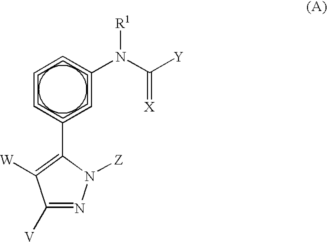
wherein:
W is lower alkyl (C₁₋₆), or halogen;
V is lower alkyl (C₁₋₆), H, or halogen;
X is either Oxygen or Sulfur;
Y is NR²R³, or (CH₂)ₘR⁴, or O(CH₂)ₙR⁴;
Z is lower alkyl (C₁₋₆);
m=0-4
n=0-4
R¹ is H or lower alkyl(C₁₋₄);
R² is H or lower alkyl(C₁₋₄);
R³ and R⁴ are independently a C₁₋₆ alkyl, or C₂₋₆ alkenyl, or cycloalkyl, or aryl group and each said group may be optionally substituted by up to four substituents in any position independently selected from CF₃, CCl₃, Me, NO₂, OH, OMe, OEt, CONR⁵R⁶, NR⁵R⁶, OCF₃, SMe, COOR⁷, SO₂NRR6, SO₃R, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl;
R⁵ and R⁶ are independently a H, or C₁₋₆ alkyl, or C₂₋₆ alkenyl, or cycloalkyl, or aryl, or CH₂ aryl group and each said group may be optionally substituted by up to four substituents in any position independently selected from CF₃, CCl₃, Me, NO₂, OH, OMe, OEt, CONR⁷R⁸, NR⁷R⁸, NHCOCH₃, OCF₃, SMe, COOR⁹, SO₃R⁷, SO₂NR⁷R⁸, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl wherein each of the C₃₋₆ cycloalkyl, C₁₋₆ alkyl, or aryl groups may be further optionally substituted by up to four substituents in any position independently selected from CF₃, CCl₃, Me, NO₂, OH, OMe, OEt, CONR⁸R⁹, NR⁸R⁹, NHCOCH₃, OCF₃, SMe, COOR⁷, SO2NR⁸R⁹, SO₃R⁷, COMe, COEt, CO-lower alkyl, SCF₃, CN, C₂₋₆ alkenyl, H, halogens, C₁₄ alkoxy, C₃₋₆ cycloalkyl, C₁₋₆ alkyl, and aryl,
or R⁵ and R⁶ may form part of a 5, 6 or 7 membered cyclic structure which may be either saturated or unsaturated and that may contain up to four heteroatoms selected from O, N or S and said cyclic structure may be optionally substituted by up to four substituents in any position independently selected from CF₃, CCl₃, Me, NO₂, OH, OMe, OEt, OCF₃, SMe, COOR⁷, SO₂NR⁸R⁹, SO₃R⁷, NHCOCH₃, COEt, COMe, or halogen;
R⁷ may be independently selected from H or C₁₋₆ alkyl;
R⁸ and R⁹ are independently a H, or C₁₋₆ alkyl, or C₂₋₆ alkenyl, or cycloalkyl, or aryl, or CH₂ aryl group and each said group may be optionally substituted by up to four substituents in any position independently selected from halogen, CF₃, OCF₃, OEt, CCl₃, Me, NO₂, OH, OMe, SMe, COMe, CN, COOR⁷, SO₃R⁷, COEt, NHCOCH₃, or aryl;
an aryl moiety can be a 5 or 6 membered aromatic heterocyclic ring (containing up to 4 hetero atoms independently selected from N, O, or S) or a 6 membered aromatic non-heterocyclic ring or a polycycle;
C₁₋₆ alkyl moieties can be straight chain or branched;
optionally substituted C₁₋₆ alkyl moieties can be straight chain or branched;
C₂₋₆ alkenyl moieties can be straight chain or branched; and
optionally substituted C₂₋₆ alkenyl moieties can be straight chain or branched, or a pharmaceutically acceptable acid addition salt thereof.

16. The method of claim 15 wherein the neuroleptic is Haloperidol and the modulator of the 5HT-2A receptor has the formula:

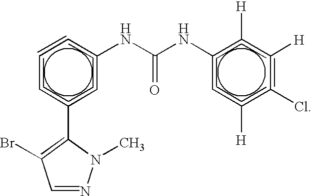

17. A method of treating psychoses in a mammal while minimizing motor-related side effects comprising administering to a subject a pharmaceutically effective amount of the composition of claim 1.

18. The method of claim 17 wherein the modulator of the 5HT-2A receptor is a compound of formula A having the formula: 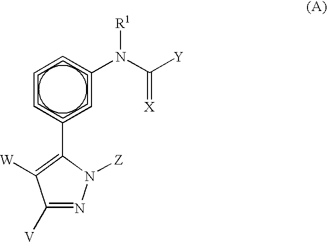
wherein:
W is lower alkyl (C₁₋₆), or halogen;
V is lower alkyl (C₁₋₆), H, or halogen;
X is either Oxygen or Sulfur;
Y is NR²R³, or (CH₂)ₘR⁴, or O(CH₂)ₙR⁴;
Z is lower alkyl (C₁₋₆);
m=0-4
n=0-4
R¹ is H or lower alkyl(C₁₋₄);
R² is H or lower alkyl(C₁₋₄);
R³ and R⁴ are independently a C₁₋₆ alkyl, or C₂₋₆ alkenyl, or cycloalkyl, or aryl group and each said group may be optionally substituted by up to four substituents in any position independently selected from CF₃, CCl₃, Me, NO₂, OH, OMe, OEt, CONR⁵R⁶, NR⁵R⁶, OCF₃, SMe, COOR⁷, SO₂NR⁵R⁶, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl;
R⁵ and R⁶ are independently a H, or C₁₋₆ alkyl, or C₂₋₆ alkenyl, or cycloalkyl, or aryl, or CH₂ aryl group and each said group may be optionally substituted by up to four substituents in any position independently selected from CF₃, CCl₃, Me, NO₂, OH, OMe, OEt, CONR⁷R⁸, NR⁷R⁸, NHCOCH₃, OCF₃, SMe, COOR⁹, SO₃R⁷, SO₂NR⁷R⁸, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl wherein each of the C₃₋₆ cycloalkyl, C₁₋₆ alkyl, or aryl groups may be further optionally substituted by up to four substituents in any position independently selected from CF₃, CCl₃, Me, NO₂, OH, OMe, OEt, CONR⁸R⁹, NR⁸R⁹, NHCOCH₃, OCF₃, SMe, COOR⁷, SO₂NR⁸R⁹, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl, or R⁵ and R⁶ may form part of a 5, 6 or 7 membered cyclic structure which may be either saturated or unsaturated and that may contain up to four heteroatoms selected from O, N or S and said cyclic structure may be optionally substituted by up to four substituents in any position independently selected from CF₃, CCl₃, Me, NO₂, OH, OMe, OEt, OCF₃, SMe, COOR⁷, SO₂NR⁸R⁹, SO₃R⁷, NHCOCH₃, COEt, COMe, or halogen;
R⁷ may be independently selected from H or C₁₋₆ alkyl;
R⁸ and R⁹ are independently a H, or C₁₋₆ alkyl, or C₂₋₆ alkenyl, or cycloalkyl, or aryl, or CH₂ aryl group and each said group may be optionally substituted by up to four substituents in any position independently selected from halogen, CF₃, OCF₃, OEt, CCl₃, Me, NO₂, OH, OMe, SMe, COMe, CN, COOR⁷, SO₃R⁷, COEt, NHCOCH₃, or aryl;
an aryl moiety can be a 5 or 6 membered aromatic heterocyclic ring (containing up to 4 hetero atoms independently selected from N, O, or S) or a 6 membered aromatic non-heterocyclic ring or a polycycle;
C₁₋₆ alkyl moieties can be straight chain or branched;
optionally substituted C₁₋₆ alkyl moieties can be straight chain or branched;
C₂₋₆ alkenyl moieties can be straight chain or branched; and
optionally substituted C₂₋₆ alkenyl moieties can be straight chain or branched, or a pharmaceutically acceptable acid addition salt thereof.

19. The method of claim 17 wherein the neuroleptic is Haloperidol and the modulator of the 5HT-2A receptor has the formula:

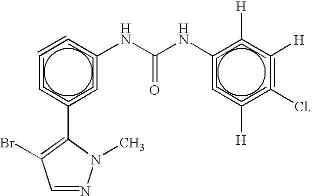

20. A method of treating psychoses in a subject while minimizing extrapyramidal motor syndrome comprising administering to a subject a pharmaceutically effective amount of the composition of claim 1.

21. The method of claim 20 wherein the modulator of the 5HT-2A receptor is a compound of formula A having the formula: 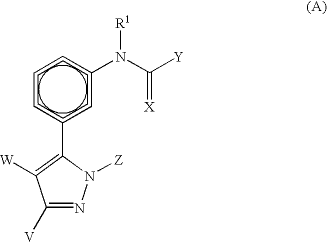
wherein:
W is lower alkyl (C₁₋₆), or halogen;
V is lower alkyl (C₁₋₆), H, or halogen;
X is either Oxygen or Sulfur;
Y is NR²R³, or (CH₂)ₘR⁴, or O(CH₂)ₙR⁴;
Z is lower alkyl (C₁₋₆);
m=0-4
n=0-4
R¹ is H or lower alkyl(C₁₋₄);
R² is H or lower alkyl(C₁₋₄);
R³ and R⁴ are independently a C₁₋₆ alkyl, or C₂₋₆ alkenyl, or cycloalkyl, or aryl group and each said group may be optionally substituted by up to four substituents in any position independently selected from CF₃, CCl₃, Me, NO₂, OH, OMe, OEt, CONR⁵R⁶, NR⁵R⁶, OCF₃, SMe, COOR⁷, SO₂NR⁵R⁶, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl;
R⁵ and R⁶ are independently a H, or C₁₋₆ alkyl, or C₂₋₆ alkenyl, or cycloalkyl, or aryl, or CH₂ aryl group and each said group may be optionally substituted by up to four substituents in any position independently selected from CF₃, CCl₃, Me, NO₂, OH, OMe, OEt, CONR⁷R⁸, NR⁷R⁸, NHCOCH₃, OCF₃, SMe, COOR⁹, SO₃R⁷, SO₂NR⁷R⁸, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl wherein each of the C₃₋₆ cycloalkyl, C₁₋₆ alkyl, or aryl groups may be further optionally substituted by up to four substituents in any position independently selected from CF₃, CCl₃, Me, NO₂, OH, OMe, OEt, CONR⁸R⁹, NR⁸R⁹, NHCOCH₃, OCF₃, SMe, COOR⁷, SO₂NR⁸R⁹, SO₃R⁷, COMe, COEt, CO-lower alkyl, SCF₃, CN, C₂₋₆ alkenyl, H, halogens, C₁₋₄ alkoxy, C₃₋₆ cycloalkyl, C₁₋₆ alkyl, and aryl,
or R⁵ and R⁶ may form part of a 5, 6 or 7 membered cyclic structure which may be either saturated or unsaturated and that may contain up to four heteroatoms selected from O, N or S and said cyclic structure may be optionally substituted by up to four substituents in any position independently selected from CF₃, CCl₃, Me, NO₂, OH, OMe, OEt, OCF₃, SMe, COOR⁷, SO₂NR⁸R⁹, SO₃R⁷, NHCOCH₃, COEt, COMe, or halogen;
R⁷ may be independently selected from H or C₁₋₆ alkyl;
R⁸ and R⁹ are independently a H, or C₁₋₆ alkyl, or C₂₋₆ alkenyl, or cycloalkyl, or aryl, or CH₂ aryl group and each said group may be optionally substituted by up to four substituents in any position independently selected from halogen, CF₃, OCF₃, OEt, CCl₃, Me, NO₂, OH, OMe, SMe, COMe, CN, COOR⁷, SO₃R⁷, COEt, NHCOCH₃, or aryl;
an aryl moiety can be a 5 or 6 membered aromatic heterocyclic ring (containing up to 4 hetero atoms independently selected from N, O, or S) or a 6 membered aromatic non-heterocyclic ring or a polycycle;
C₁₋₆ alkyl moieties can be straight chain or branched;
optionally substituted C₁₋₆ alkyl moieties can be straight chain or branched;
C₂₋₆ alkenyl moieties can be straight chain or branched; and
optionally substituted C₂₋₆ alkenyl moieties can be straight chain or branched,
or a pharmaceutically acceptable acid addition salt thereof.

22. The method of claim 20 wherein the neuroleptic is Haloperidol and the modulator of the 5HT-2A receptor has the formula:

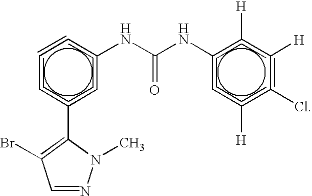

23. The method of any one of claims 11, 13, 14, 16, 17, 19 or 20 further comprising the step of identifying a subject, said subject being in need of treatment of a psychosis susceptible to undesired side effects, wherein said identifying step is performed prior to administering to said subject said pharmaceutically effective amount of said composition.
